(19) United States
(12) Patent Application Publication
(10) Pub. No.: US 2025/0228598 A1
(43) Pub. Date: Jul. 17, 2025

(54) METHODS FOR ESTIMATING AN AMOUNT OF CRYOGEN IN A VESSEL, AND RELATED DEVICES AND SYSTEMS

(71) Applicant: HOLOGIC, INC., MARLBOROUGH, MA (US)

(72) Inventors: Edward David PIEDRA, Chicopee, MA (US); Sophia Marie TRIANTIS, Cambridge, MA (US)

(73) Assignee: HOLOGIC, INC., MARLBOROUGH, MA (US)

(21) Appl. No.: 19/016,055

(22) Filed: Jan. 10, 2025

Related U.S. Application Data
(60) Provisional application No. 63/619,907, filed on Jan. 11, 2024.

(51) Int. Cl. A61B 18/02; A61B 18/00; A61B 90/00; F17C 13/02; G01F 23/18

(52) U.S. Cl. CPC A61B 18/02; F17C 13/025; G01F 23/18; A61B 2018/00577; A61B 2018/00642; A61B 2018/00744; A61B 2090/064; F17C 2205/0326; F17C 2205/0338; F17C 2223/0161; F17C 2227/0157; F17C 2250/0417; F17C 2250/043; F17C 2250/0694; F17C 2270/02

(57) ABSTRACT

A method for estimating an amount of cryogenic fluid in a cryogenic fluid supply vessel comprises releasing pressure from the cryogenic fluid supply vessel at a predetermined frequency via a control valve. The method also comprises detecting a disturbance in an output signal of a pressure feedback sensor. The disturbance occurs at the predetermined frequency. The method additionally comprises determining a magnitude of the disturbance in the output signal of the pressure feedback sensor and estimating the amount of the cryogenic fluid in the cryogenic fluid supply vessel based on the magnitude of the disturbance in the output signal of the pressure feedback sensor.

### CROSS-REFERENCE TO RELATED APPLICATIONS

This application claims priority to U.S. Provisional Application No. 63/619,907, filed Jan. 11, 2024, the entire contents of which are incorporated by reference herein.

### TECHNICAL FIELD

The present disclosure generally relates to the field of cryoablation devices and systems. More specifically, the disclosure relates to methods and systems for estimating an amount of cryogen contained in a cryogenic fluid supply vessel prior to, during, and/or after a cryoablation procedure.

### INTRODUCTION

The section headings used herein are for organizational purposes only and are not to be construed as limiting the subject matter described in any way.

Cryogenic delivery systems, including systems with cryosurgical probes, are used to destroy diseased or abnormal tissue cells to treat a variety of medical conditions. When cryogenic liquids such as liquid nitrogen are used with a cryosurgical probe, tissues adjacent to the probe quickly freeze, causing the tissue to die, after which it is absorbed by the body, expelled by the body, or sloughed off. Cryogenic delivery systems can be used to treat, among other things, skin cancer, skin lesions, breast tumors (both benign and cancerous), prostate cancer and benign prostate disease, liver tumors and liver cancer, glaucoma and other eye diseases, and other conditions.

Cryogenic delivery systems such as cryoablation devices receive a supply of a cryogenic fluid, such as liquid nitrogen or other cryogenic liquid, from a fluid supply vessel. The cryogenic fluid is consumed during a cryogenic procedure and, once the fluid supply vessel is empty, further operation of the delivery system requires the empty fluid supply vessel to be replaced with another vessel containing cryogenic fluid. Devices and methods have therefore been developed to determine the amount of cryogenic fluid that is contained within a fluid supply vessel (i.e., to confirm that there is enough cryogenic fluid within the fluid supply vessel to complete a procedure) prior to starting a cryogenic procedure. Such devices and methods, for example, may utilize a load cell to detect the volume of cryogenic fluid within the fluid supply vessel (e.g., within a Dewar when the Dewar is first placed in a Dewar chamber of the cryogen delivery system), to confirm that the volume of cryogenic fluid is sufficient prior to starting the procedure. In this manner, once the volume of cryogenic fluid within the vessel is confirmed, the cryogenic delivery system may begin a cryogenic procedure. To initiate the cryogenic procedure, however, the cryogenic delivery system must generally form a strong seal with the fluid supply vessel (e.g., the Dewar containing the cryogen is generally lifted to form a seal against an O-ring assembly in the Dewar chamber of the delivery system) to prevent cryogenic fluid from escaping from the fluid supply vessel and to maintain pressure within the Dewar during the procedure. Thus, once the procedure begins and this seal is formed, since the force of forming the seal may be seen in the load cell, possibly saturating, or corrupting the load signal, the load cell may lose its ability to accurately estimate the volume of cryogenic fluid within the vessel throughout the remainder of the procedure.

While this may not pose a problem for all cryogenic procedures, as for example some cryoablation procedures require that a cryogenic delivery system automatically run for a preset amount of time using a preestablished amount of cryogenic fluid that is sufficient to complete the procedure (i.e., to operate in an automatic mode once a procedure begins), other cryoablation procedures require that a user (e.g., a surgeon or radiologist) manually control the freeze/thaw times during the procedure (i.e., to operate in a manual mode), thereby requiring a real time assessment (and not just an initial assessment) of how much cryogenic fluid has been consumed and whether or not there is enough cryogenic fluid left to complete the procedure. For example, such cryoablation procedures can include alternating cycles of freezing and thawing intervals, such as two freezing cycles with a passive thawing interval in between the freezing cycles. If the cryogenic fluid is consumed prior to the desired end time of the procedure, the tissue mass desired to be frozen and destroyed may not be sufficiently cooled, and the procedure may not be completed successfully or to the degree desired or expected.

Furthermore, even for cryoablation procedures utilizing an automatic mode, in the event of a failure mode (i.e., in which the workflow is not followed, and the procedure needs to be restarted) or any other missteps in which some of the cryogenic fluid may be inadvertently used, a user may also need to determine how much cryogenic fluid is available after a procedure begins.

There is therefore a need for methods and devices for accurately estimating an amount of cryogenic fluid within a fluid supply vessel, such as, for example, a volume of cryogenic fluid within the fluid supply vessel, even after a cryogenic procedure has started, throughout the entirety of the cryogenic procedure, and/or after the procedure is over (e.g., to determine if a fluid supply vessel can be reused in another procedure without being refilled). There is a further need to provide this information to the operator in a manner in which the operator can determine whether the available amount of cryogenic fluid present in the fluid supply vessel is sufficient to complete or initiate a planned procedure.

### SUMMARY

The present disclosure addresses one or more of the above-mentioned problems and/or achieves one or more of the above-mentioned desirable features. Other features and/or advantages may become apparent from the description which follows.

In accordance with various exemplary embodiments of the present disclosure, a method for estimating an amount cryogenic fluid in a cryogenic fluid supply vessel comprises releasing pressure from the cryogenic fluid supply vessel at a predetermined frequency via a control valve. The method also comprises detecting a disturbance in an output signal of a pressure feedback sensor. The disturbance occurs at the predetermined frequency. The method additionally comprises determining a magnitude of the disturbance in the output signal of the pressure feedback sensor and estimating the amount of the cryogenic fluid in the cryogenic fluid supply vessel based on the magnitude of the disturbance in the output signal of the pressure feedback sensor.

In accordance with various additional exemplary embodiments of the present disclosure, a method for estimating an amount of liquid nitrogen in a cryogenic fluid supply vessel during a cryoablation procedure comprises supplying a constant pressure to the cryogenic fluid supply vessel via a compressor of a cryogen delivery system. The method also comprises regulating a pressure in the cryogenic fluid supply vessel by releasing the pressure from the cryogenic fluid supply vessel. The pressure from the cryogenic fluid supply vessel comprises a combination of the constant pressure from the compressor and a fluid pressure from a volume of liquid nitrogen in the cryogenic fluid supply vessel. The method also comprises detecting a ripple in an output signal of a pressure feedback sensor of the cryogen delivery system. The ripple occurs at a frequency corresponding to a rate of pressure release from the cryogenic fluid supply vessel. The method further comprises determining a magnitude of the ripple in the output signal of the pressure feedback sensor and estimating a remaining amount of the liquid nitrogen in the cryogenic fluid supply vessel based on the magnitude of the ripple in the output signal of the pressure feedback sensor.

In accordance with various further exemplary embodiments of the present disclosure, a method of ablating a target tissue during a cryoablation procedure comprises inserting a cryogenic probe of a cryogen delivery system into a treatment region of a patient. The cryogenic probe is in fluid communication with a cryogenic fluid supply vessel containing liquid nitrogen. The method also comprises supplying a constant pressure to the cryogenic fluid supply vessel via a compressor of the cryogen delivery system during a freeze cycle of the cryoablation procedure to supply the liquid nitrogen to the cryogenic probe. The method additionally comprises regulating a pressure in the cryogenic fluid supply vessel by releasing the pressure from the cryogenic fluid supply vessel. The pressure from the cryogenic fluid supply vessel comprises a combination of the constant pressure from the compressor and a fluid pressure from a volume of liquid nitrogen in the cryogenic fluid supply vessel. The method also comprises monitoring an amount of liquid nitrogen remaining in the cryogenic fluid supply vessel during the freeze cycle by detecting a ripple in an output signal of a pressure feedback sensor of the cryogen delivery system, the ripple occurring at a frequency corresponding to a rate of pressure release from the cryogenic fluid supply vessel, determining a magnitude of the ripple in the output signal of the pressure feedback sensor, and based on the magnitude of the ripple in the output signal of the pressure feedback sensor, estimating an amount of the liquid nitrogen remaining in the cryogenic fluid supply vessel.

Additional objects and advantages will be set forth in part in the description which follows, and in part will be obvious from the description, or may be learned by practice of the present teachings. At least some of the objects and advantages of the present disclosure may be realized and attained by means of the elements and combinations particularly pointed out in the appended claims.

Both the foregoing general description and the following detailed description are exemplary and explanatory only and are not restrictive of the present disclosure and claims, including equivalents. The present disclosure and claims, in their broadest sense, could be practiced without having one or more features of these exemplary aspects and embodiments.

### BRIEF DESCRIPTION OF THE DRAWINGS

The accompanying drawings, which are incorporated in and constitute a part of this specification, illustrate some exemplary embodiments of the present disclosure and together with the description, serve to explain certain principles. These drawings depict only typical embodiments of the disclosed inventions and are not therefore to be considered limiting of its scope. In the drawings:

FIG. 1 is a perspective view of an exemplary embodiment of a cryogenic delivery system console in accordance with the present disclosure.

FIG. 2 is a high-level, block diagrammatic view of an exemplary control system of a cryogenic delivery system in accordance with the present disclosure.

FIG. 3 is a block diagrammatic view of an exemplary pressure control loop of the control system of FIG. 2.

FIG. 4 illustrates a visual interface of a cryogenic delivery system according to an exemplary embodiment of the present disclosure.

FIG. 5A is a graph illustrating exemplary outputs from the pressure regulating system of FIG. 3, at the start of a cryosurgical procedure.

FIG. 5B is a graph illustrating exemplary outputs of the pressure regulating system of FIG. 3, during a cryosurgical procedure.

FIG. 5C is a partial, enlarged view of a sensor output of FIG. 5B.

FIG. 5D is a graph illustrating exemplary outputs of the pressure regulating system of FIG. 3, at the end of a cryosurgical procedure.

FIG. 6 is a flow chart showing a workflow for a cryosurgical procedure according to an exemplary embodiment of the present disclosure.

### DETAILED DESCRIPTION OF THE ILLUSTRATED EMBODIMENTS

Exemplary embodiments of the present disclosure include cryogen delivery systems configured for medical treatment or surgical use, such as cryosurgical or cryoablation systems, and which utilize methods and systems for estimating an amount of cryogenic fluid that is available for use during a procedure in which it is employed. The cryogen delivery systems may include, for example, features configured to provide information to an operator of the cryogenic delivery system regarding an amount of cryogenic fluid present in a cryogenic fluid supply vessel, such as a Dewar, flask, or other thermal container, prior to initiation of a cooling operation or procedure.

In some embodiments, for example, once an operator initiates a cooling operation or procedure (or reinitiates a cooling operation or procedure, such as, for example, after an error), cryogenic delivery systems employing methods in accordance with the present disclosure may monitor a pressure output from a cryogenic fluid supply vessel, such as, for example, a Dewar containing liquid nitrogen for use during the procedure, and estimate a volume of the liquid nitrogen within the Dewar based on a disturbance in the monitored pressure output. The cryogenic delivery systems may then provide information to the operator of the cryogenic delivery system regarding the volume of liquid nitrogen in the Dewar, to allow the operator to determine whether the procedure should continue or whether the Dewar needs to be replaced (i.e., with a new Dewar containing a greater amount of liquid nitrogen).

In some embodiments, upon initiation of a cooling operation or procedure, a control system of a cryogenic delivery system may begin to supply pressure to the cryogenic fluid supply vessel (e.g., the Dewar). To regulate the pressure of the cryogenic fluid supply vessel during the procedure, the control system may also release pressure from the cryogenic fluid supply vessel, for example, at a predetermined frequency via a control valve of the cryoablation system. For example, as will be described further below, a compressor pump of the cryoablation system may supply a constant pressure (Ps) to the Dewar, which combines with a fluid pressure (P_{LN2}(t)) from the cryogenic fluid within the Dewar (e.g., the liquid nitrogen (LN₂) within the Dewar), and the control system may be configured to release this combined pressure P_{D} to prevent a pressure buildup within the Dewar. In some embodiments, the control system may also be configured to monitor the pressure within the Dewar (e.g., the combined pressure P_{D}), via, for example, a pressure feedback sensor of the cryoablation system, and to detect a disturbance in an output signal of the pressure feedback sensor that is associated, for example, with the pressure release from the Dewar. In other words, the control system may be configured to detect a disturbance in the output signal of the pressure feedback sensor that occurs at the predetermined frequency of pressure release. In such embodiments, the control system may be further configured to determine a magnitude of the disturbance in the output signal of the pressure feedback sensor and to estimate the volume of the cryogenic fluid (e.g., the LN2) in the Dewar based on the magnitude of the disturbance in the output signal of the pressure feedback sensor.

The sensor can, for example, be operably coupled to the control system of the cryogenic delivery system, and the information from the sensor can be processed and provided to the operator of the cryogenic delivery system, e.g., via a visual interface such as a display, an audio indicator, or other indicator. The indicator can be text, a color, an icon, or a combination of all three on a display, and/or a warning light located on the console. Additionally, or alternatively, the indicator may include an audio component. The control system can be configured to provide the information from the sensor in terms of a volume of cryogenic fluid contained in the vessel (e.g., Dewar), a weight of the cryogen contained in the vessel, a fractional indication of cryogenic fluid relative to a full container of cryogen, an estimated run time available, or other parameter. For example, in some embodiments, the cryogenic delivery system can use the information indicative of the amount of cryogenic fluid in the fluid supply vessel to determine, for example, a length of time the delivery system can run based on the amount of cryogenic fluid remaining in the vessel and a flow rate of the delivery system. Based on this determination, and dependent upon other input provided by an operator of the cryogenic delivery system, the system can provide feedback to the system operator regarding a procedure to be initiated. The type of feedback may be general, such as an indication that sufficient cryogenic fluid is present, an indication that insufficient cryogenic fluid is present or instructions to connect a new (or different) cryogenic fluid supply vessel or to refill the existing cryogenic fluid supply vessel, or the feedback may be more specific, such as an indication of an amount of time the system can run given the amount of cryogenic fluid present, instructions to replace the cryogenic fluid supply vessel during a thawing phase of the procedure, or other feedback to guide the operator's use of the cryogenic delivery system.

According to exemplary embodiments of the present disclosure, the pressure feedback sensor can be or can include a strain gauge sensor, a sensor including a plurality of strain gauges, such as, for example, a piezoelectric transducer, or other sensor. The sensor can be operably coupled to the control system, which can be or include one or more processors operably coupled with electronic storage elements such as random-access memory, read-only memory, or other storage mediums, and programmed with software to control the various functions of the cryogen delivery system.

In alternate example embodiments, one or more sensors may be used in a storage area of the cryogenic delivery system, in which the storage area is configured to support and store multiple cryogenic fluid supply vessels (e.g., multiple Dewars). Each portion of the storage area that is configured to support a cryogenic fluid supply vessel can, for example, have an individual pressure feedback sensor associated with a respective cryogenic fluid supply vessel, wherein the control system is configured to monitor the output of each pressure feedback sensor, to detect a disturbance in an output signal of each pressure feedback sensor (i.e., associated with a pressure release from a respective Dewar), to determine a magnitude of the disturbance in the output signal of each pressure feedback sensor, and to estimate the volume of the cryogenic fluid (e.g., the LN2) in a respective Dewar based on the magnitude of the disturbance. In such embodiments, in addition to the type of information indicated above, the control system may provide information to the operator of the system such as directing the loading/installation of a fluid supply vessel from a particular storage area based on the procedure parameters input by the operator.

Exemplary embodiments of the present disclosure can be used with, for example, cryogen delivery systems described in connection with U.S. Pat. No. 8,092,448, filed Apr. 27, 2007, and International Patent Application Publication No. WO 2023/097220, filed Nov. 22, 2022, the entire contents each of which are incorporated by reference herein.

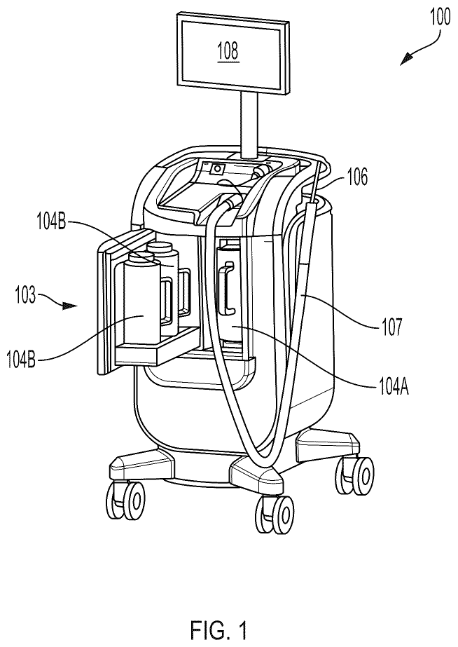

Referring now to FIG. 1, a cryogen delivery system 100 according to the present disclosure is shown. As shown in FIG. 1, the cryogen delivery system 100 can be in the form of a wheeled console to facilitate ease of use and portability. In some embodiments, the cryogen delivery system 100 can be configured to perform, for example, a cryosurgical procedure such as, for example, a cryoablation procedure. The cryogen delivery system 100 comprises a probe or needle 106, a cryogenic fluid supply hose 107 to which the probe 106 is coupled, and a cryogenic fluid supply vessel (e.g., a Dewar) 104A. The cryogenic fluid supply hose 107 is configured to receive a flow of cryogenic fluid from the cryogenic fluid supply vessel 104A for delivery via the probe 106 to a treatment location. The cryogen delivery system 100 includes a storage area 103 for storage of additional cryogenic fluid supply vessels 104B. The cryogen delivery system 100 also includes a visual interface, such as a display 108, configured to communicate information to the operator of the cryogen delivery system 100 regarding the operational state of the cryogen delivery system 100. In some embodiments, the display 108 may comprise touchscreen controls to enable the operator to alter or input operational parameters and otherwise control the cryogen delivery system 100.

According to various embodiments of the present disclosure, the display 108 may also be configured to display information regarding an amount of cryogenic fluid, such as, for example, a volume of cryogenic fluid, present or available in the cryogenic fluid supply vessel 104A. Such information can be provided directly as an available volume of cryogenic fluid, or converted into an available weight of cryogenic fluid, a fraction of a full volume or weight (i.e., where the fraction represents a percentage of a full or unused amount of cryogenic fluid), or other indication. Alternatively, instead of providing an amount that is available, the indication may be presented as an amount remaining in a fluid supply vessel, where the cryogenic fluid in fluid supply vessels used in prior procedures can be partially depleted. In some embodiments, the display may provide an expected available possible runtime based on the available amount of cryogen.

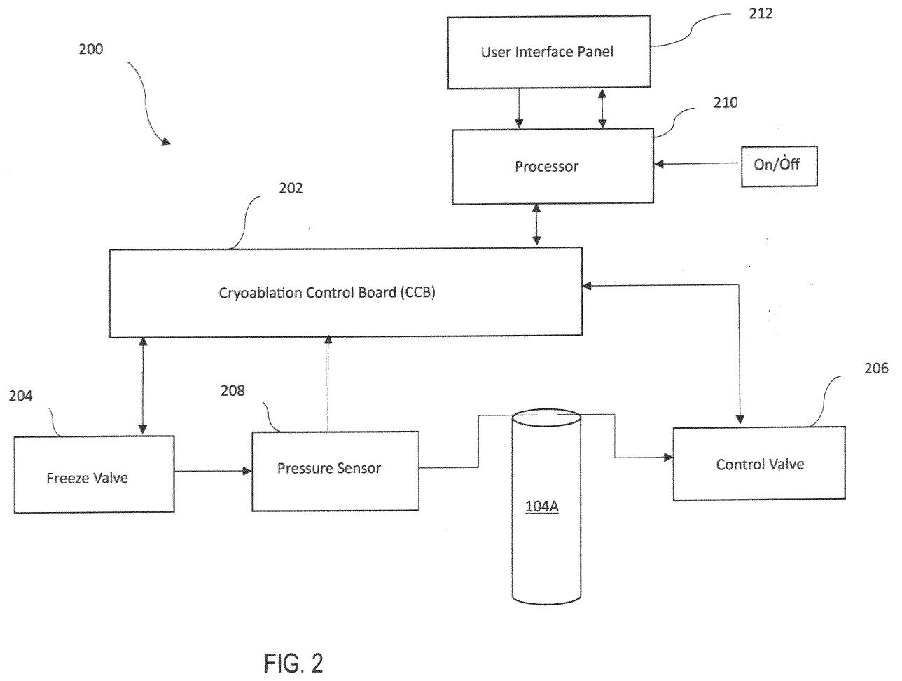

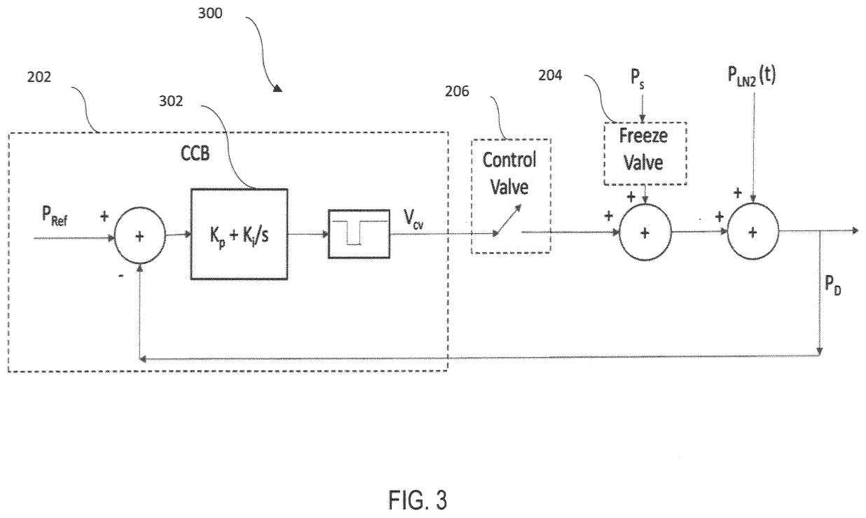

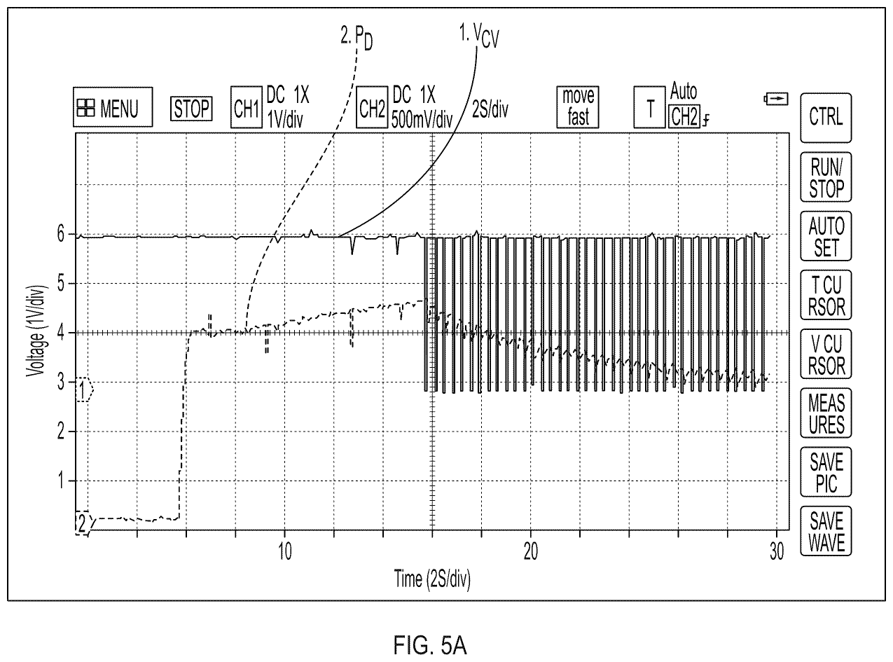

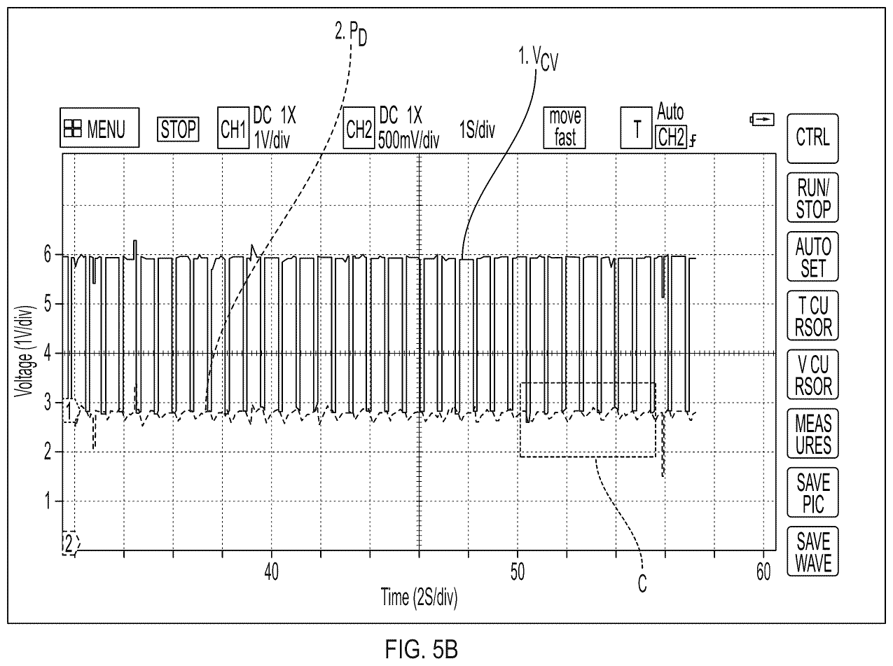

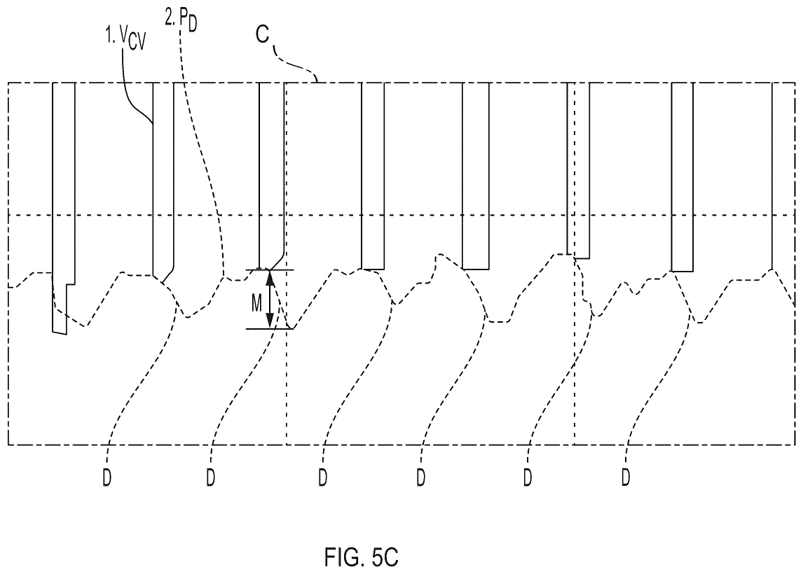

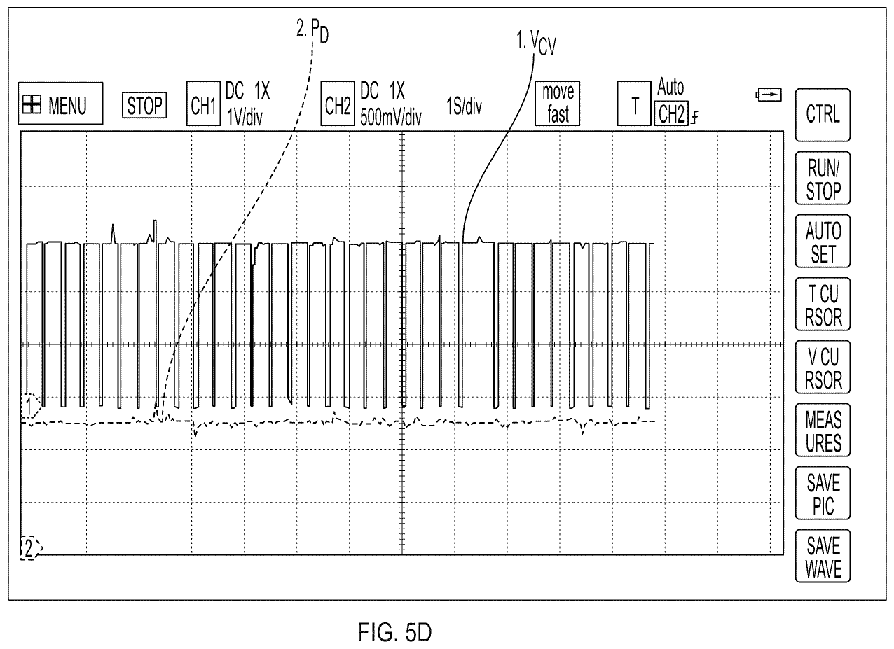

Referring now to FIGS. 2 and 3, high-level, block diagrammatic views of an exemplary control system 200 and pressure control loop 300 of a cryogenic fluid delivery system, such as, for example, the cryogen delivery system 100, housing the cryogenic fluid supply vessel 104A, is shown. In some embodiments, as discussed above, the cryogen delivery system 100 is configured to perform a cryosurgical procedure such as, for example, a cryoablation procedure, utilizing pressurized liquid nitrogen (LN2) that is held within the cryogenic fluid supply vessel (e.g., the Dewar) 104A, and which is controlled via a cryoablation control board (CCB) 202 of the control system 200. Upon initiation of a cryoablation procedure, the CCB 202, for example, is configured to open a freeze valve 204, thereby allowing the pressure P_{D} in the Dewer 104A (i.e., Dewar pressure P_{D}) to build to an acceptable pressure to begin the procedure (see, e.g., FIG. 5A). As discussed above, the freeze valve 204 is operably coupled to a compressor pump (not shown), which is configured to supply a constant pressure Ps to the Dewar 104A. When the compressor pump is turned on and starts to supply pressure to the Dewar 104A, this constant pressure Ps combines with a fluid pressure (P_{LN2}(t)) from the cryogenic fluid within the Dewar 104A (e.g., the liquid nitrogen (LN2) within the Dewar) to provide the Dewar pressure P_{D} (i.e., which is monitored by a pressure feedback sensor 208).

To prevent the Dewar pressure P_{D} from getting too high, the CCB 202 is further configured to regulate the Dewar pressure P_{D} to a preestablished level or reference pressure P_{Ref} through operation of a pressure control loop 300. In some embodiments, for example, the CCB 202 is configured to release pressure from the Dewar 104A at a predetermined frequency via a control valve 206. In other words, the CCB 202 may open and close (or toggle) the control valve 206 (e.g., via a pulse-width modulation (PWM) voltage command V_{CV}) at a given rate to maintain the Dewar pressure P_{D} around the reference pressure P_{Ref}. As illustrated in FIG. 3, the CCB 202 may employ a proportional integral (PI) regulator 302 (utilizing proportional and integral gains Kₚ and Kᵢ) to control and adjust the Dewar pressure P_{D} to follow the reference pressure P_{Ref}, as would be understood by those of ordinary skill in the art.

In some exemplary embodiments, the CCB 202 is configured to regulate the Dewar pressure P_{D} to a reference pressure P_{Ref} of about 30 psi through operation of the pressure control loop 300 by opening/closing (or toggling) the control valve 206 at a rate of about 3.33 Hz. In such embodiments, as will be discussed further below with reference to FIG. 5A, upon initiation of a cryoablation procedure, the CCB 202, is configured to open the freeze valve 204 and allow the pressure P_{D} in the Dewer 104A (i.e., Dewar pressure P_{D}) to build for about 10 seconds before it starts to toggle the control valve 206 at a rate of about 3.33 Hz (i.e., to maintain the Dewar pressure P_{D} at about 30 psi). Those of ordinary skill in the art will understand that a Dewar pressure P_{D} of 30 psi and a toggle frequency of 3.33 Hz are only one set of exemplary reference settings and that the present disclosure can be applied to other reference Dewar pressures/toggle frequencies without departing from the present disclosure and claims.

To regulate the Dewar pressure P_{D}, the CCB 202 is also configured to monitor the Dewar pressure P_{D} via a pressure feedback sensor 208, for example, by monitoring an output signal of the pressure feedback sensor 208. In some embodiments, the CCB 202 is also configured to detect a disturbance in the output signal of the pressure feedback sensor 208. With reference to FIGS. 5A, 5B, 5C, and 5D for example, which illustrate exemplary outputs from the pressure control loop 300 at the beginning, during, and end of a cryosurgical procedure, including a channel 1 correlating to the PWM voltage command V_{CV} output (i.e., supplied by the CCB to the control valve 206) and a channel 2 correlating to the Dewar pressure P_{D} output (i.e., as measured by the pressure feedback sensor 208), there is a visible disturbance in the Dewar pressure P_{D} output whenever the voltage command V_{CV} output fluctuates. In other words, as best illustrated perhaps in the enlarged view of FIG. 5C, whenever the CCB 202 toggles the control valve 206 by pulsing the voltage command V_{CV}, for example, by about 3.33 Hz, there is a ripple in the measured Dewar pressure P_{D} output. In accordance with the present disclosure, the CCB 202 is configured to detect this disturbance (or ripple) D in the output signal of the pressure feedback sensor 208 (which occurs at the predetermined frequency of pressure release from the Dewar 104A, in this case, at about 3.33 Hz). In other words, in some embodiments, the CCB 202 is configured to detect a 3.33 Hz disturbance D in the output signal of the pressure feedback sensor 208.

The present disclosure further contemplates that the control system 200 may determine an amount of cryogenic fluid in the Dewar 104A based on this disturbance (or ripple) D in the output signal of the pressure feedback sensor 208. For example, although not wishing to be bound by a particular theory, it has been found that a magnitude M of a respective disturbance D correlates directly with a volume of cryogenic fluid in the Dewar 104A, such that as the volume of cryogenic fluid in the Dewar 104A decreases a magnitude M of a disturbance D in the output signal of the pressure feedback sensor 208 decreases, until the magnitude M zeros out (i.e., the peak to peak disturbance D is no longer visible), such that the output signal of the pressure feedback sensor 208 is approaching a flat line as illustrated in FIG. 5D (i.e., thereby indicating that the Dewar 104A is approximately empty).

In accordance with the present disclosure, the CCB 202 is therefore further configured to determine a magnitude M of the disturbance D in the output signal of the pressure feedback sensor 208 and to estimate a volume of the cryogenic fluid (e.g., the LN2) in the Dewar 104A based on the magnitude M of the disturbance D in the output signal of the pressure feedback sensor 208. This proportional relationship between the magnitude M of the disturbance D and the volume of cryogenic fluid in the Dewar can be leveraged in various embodiments through the use of software applications or algorithms to permit the amount of cryogenic fluid in the Dewar to be identified at any point prior to, during, and/or after a procedure. For example, in some embodiments, to determine the magnitude M of a respective disturbance D, the CCB 202 is configured to extract the respective disturbance D from the output signal of the pressure feedback sensor 208 and apply a min/max detection algorithm to the extracted disturbance D. In some other example embodiments, to determine the magnitude M of a respective disturbance D, the CCB 202 is configured to extract the respective disturbance D from the output signal of the pressure feedback sensor 208 and apply a discrete Fourier transform (DFT) to the extracted disturbance D. As discussed above, the CCB 202 may then use the determined magnitude M to determine a volume of cryogenic fluid in the Dewar 104A, for example, by referencing a lookup table, applying a linear curve or a polynomial fit, or other known correlation method. As software applications involving a min/max detection algorithm, a DFT, look up tables, and linear curves/polynomial fits, are well known in the art, the specifics of such mathematical applications will not be discussed in detail herein. Those of ordinary skill in the art will understand that any number of algorithms can be employed to the extracted disturbances D to determine the magnitudes M of the disturbances and correlate the determined magnitudes M with a volume of cryogenic fluid in the Dewar.

Those of ordinary skill in the art will further understand that the above-described system 200 and loop 300 are exemplary only and that further modifications and alternative examples will be apparent in view of the disclosure herein. For example, each of the control system 200 and the pressure control loop 300 may include additional components and/or system configurations that were omitted from FIGS. 2 and 3 and description thereof for clarity of operation. Accordingly, the above description is to be construed as illustrative only and is for the purpose of teaching those skilled in the art the general manner of carrying out the volume estimation methods disclosed in the present teachings. Thus, changes may be made in the elements described with relation to the system 200 and loop 300 without departing from the scope of the present teachings and claims.

The cryogen volume estimation methods and systems contemplated by the present disclosure enable reliable, real time volume estimation of the cryogenic fluid (e.g., the LN2) in the fluid supply vessel (e.g., Dewar) 104A, and available to the cryogen delivery system 100, at any time prior to, during, and/or after a cryogenic procedure, and are not limited to, for example, the very beginning of a procedure (e.g., prior to the fluid supply vessel 104A forming a seal with the cryogen delivery system 100), and do not require a pause in a procedure (e.g., allowing the seal between the fluid supply vessel 104A and the cryogen delivery system 100 to be broken in order to make an estimation). Such methods and systems may, therefore, allow the cryogen delivery system 100 to provide a real time assessment (and not just an initial assessment) of how much cryogen has been consumed and whether or not there is enough cryogen left to complete a procedure, when, for example, a user (e.g., surgeon) manually controls the freeze/thaw times during the procedure (i.e., operates the cryogen delivery system 100 in a manual mode). For example, such cryoablation procedures can include alternating cycles of freezing and thawing intervals, such as two freezing cycles with a passive thawing interval in between the freezing cycles. If the cryogen is consumed prior to the desired end time of the procedure, the tissue mass desired to be frozen and destroyed may not be sufficiently cooled, and the procedure may not be completed successfully or to the degree desired or expected.

The contemplated systems and methods are also advantageous for cryoablation procedures utilizing an automatic mode (in which the cryogen delivery system 100 is configured to automatically run for a preset amount of time using a preestablished amount of cryogen that is sufficient to complete the procedure). For example, in the event of an error or failure (i.e., in which the workflow is not followed or there is a system failure (e.g., a blockage detection, spike in pressure, or an inability of the system to detect the flow of LN2), and the procedure needs to be restarted), and/or any other missteps in which some of the cryogen may be inadvertently used, a surgeon may also need to determine how much cryogen is available after a procedure begins.

Furthermore, the contemplated systems and methods may provide the cryogen delivery system 100 with a safety or redundancy feature, which allows the cryogen delivery system 100 to verify (i.e., double check) a previously determined cryogen volume measurement to ensure that an adequate amount of cryogen is available to the system throughout a procedure. Once a cryoablation procedure begins, the cryogen delivery system 100 may, for example, utilize the contemplated real time volume estimation methods to verify a previous estimate, determined for example using a different method, prior to pressurization of the fluid supply vessel (e.g., Dewar) 104A. In some embodiments, for example, the contemplated volume estimation methods and systems may be used in conjunction with the methods and systems described in connection with International Patent Application Publication No. WO 2023/097220, incorporated herein by reference above. In some additional embodiments, the cryogen delivery system 100 may be configured to continuously utilize the contemplated real time volume estimation methods throughout a procedure to provide a running, real time assessment of the amount of cryogen available to the system 100, and to alert a user of the system 100 once the estimated volume reaches a threshold level.

Referring again to FIG. 2, according to embodiments of the present disclosure, the control system 200 of the cryogen delivery system 100 can comprise a pressure feedback sensor 208 configured to generate an output signal indicative of the overall pressure P_{D} in the cryogenic fluid supply vessel (e.g., Dewar) 104A, and a magnitude M of a disturbance D in this signal can be correlated to a volume of the cryogenic fluid (e.g., LN₂) in the cryogenic fluid supply vessel 104A. The cryoablation control board (CCB) 202 is configured to receive the signal from the sensor 208 and process the signal, for example, via a processor 210, to provide information to the operator of the cryogen delivery system 100 regarding the amount of cryogenic fluid present in the cryogenic fluid supply vessel 104A. As discussed above, such processing can include, for example, applying min/max detection algorithms, discrete Fourier transforms (DFTs), look up tables, and/or curve fits to the data.

The determined volume of the cryogenic fluid present or available in the cryogenic fluid supply vessel 104A can be displayed to the operator, e.g., via a user interface panel 212 (e.g., of display 108 of FIG. 1) or other visual or audio indicators. Additionally, the determined volume of the cryogen present or available in the cryogenic fluid supply vessel 104A can be converted to an available run time, so that the operator can decide whether the available amount of cryogenic fluid is sufficient for a planned procedure. The available run time can be calculated as a function of the amount of cryogenic fluid available and one or more known, constant flow rates of the probe 106. Additionally, or alternatively, the display can provide a binary go/no go indicator based on a time required for a procedure chosen or input by the operator and the determined available amount of cryogenic fluid.

In some embodiments, as described in connection with International Patent Application Publication No. WO 2023/097220, incorporated herein by reference above, the cryogen delivery system 100 can include an automated cryogenic fluid supply vessel switching system configured to automatically switch between cryogenic fluid supply vessels 104A and 104B (FIG. 1) based on a determined volume of cryogen available in each of the cryogenic fluid supply vessels 104A and 104B. For example, in some example embodiments, the storage area 103 (FIG. 1) can include an automated system configured to automatically reposition each of cryogenic fluid supply vessels 104A and 104B in turn on the moveable support structure (not shown). The automated movement of the system can be driven by the control system 200 of the cryogen delivery system 100 or automated movement may be initiated, via user input, based on the determined volumes of cryogen in each of the cryogenic fluid supply vessels 104A and 104B.

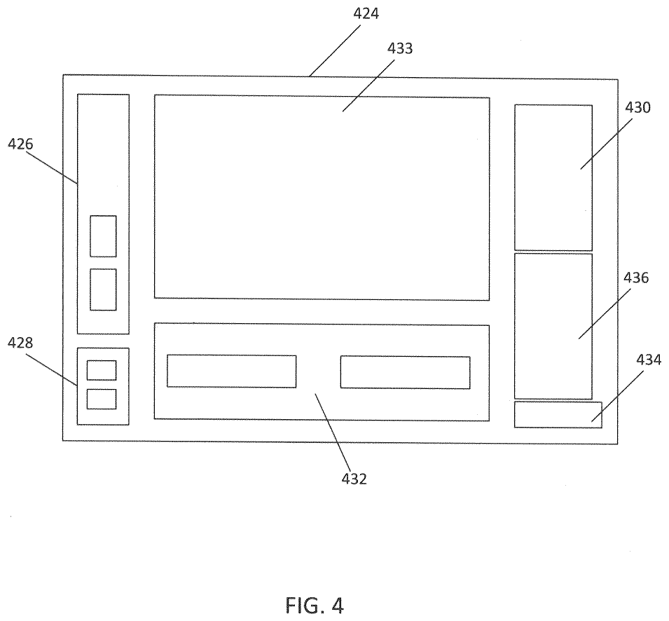

FIG. 4 is a diagram view of a display 424 of a cryogen delivery system, such as, for example, the cryogen delivery system 100 of FIG. 1, according to an exemplary embodiment of the present disclosure. In the embodiment of FIG. 4, the display 424 comprises a touchscreen with various controls related to the operation of the cryogen delivery system 100. For example, the display 424 can include, among other things, vessel controls 426 for raising and lowering the cryogenic fluid supply vessel, probe controls 428 for locking out use of the probe 106 (FIG. 1) to prevent inadvertent actuation of the cryogen delivery system, a status area 430 for communicating various messages to the operator, a mode selection area 432, a shutdown button 434, and a general display field 433 to display other information used for operation of the cryoablation system.

In some embodiments, the display 424 can include a cryogen status indicator area 436 that is configured to display, based on the processed signal from the pressure feedback sensor 208 (FIG. 2), the estimated volume of cryogen available in the cryogenic fluid supply vessel 104A (FIGS. 1 and 2). As discussed above, the cryogen status indicator area 436 can provide the information as an available volume, a maximum procedure run time, and/or a binary go/no-go indicator that indicates whether the amount of cryogen present/available is sufficient to complete a procedure chosen by the operator. The cryogen status indicator area 436 can be configured to display the information regarding the available volume of cryogen numerically, e.g., by actual volumes, by percentages (based on volume of full fluid supply vessel), or can be configured to display the information graphically, such as with a bar graph, gauge, or other graphical representation. Further, the display can be configured such that the operator can choose a desired type of information display. That is, in some embodiments, the operator can choose whether to view the information as an available volume, maximum run time, etc. The cryogen status indicator area 436 can be configured to display the information regarding the available volume of cryogen via text, colors, icons, warning lights (flashing or steady state) on the display or on a portion of the console (e.g., console 100 shown in FIG. 1), or any combination thereof. The visual indicators may be combined with audio indicators such as speech reciting displayed text indicators, beeping, or other audible indicators.

While the exemplary embodiment of the cryogen delivery system 100 is configured to display the information regarding available cryogen via a display 108, such as, for example, the display 424, other embodiments can include other indicators, such as visual indicators not associated with the display 424, audio indicators, or other combinations of indicators. Further, in other exemplary embodiments, the various controls and information areas can be relocated, reconfigured, or combined in different ways without departing from the scope of the present disclosure.

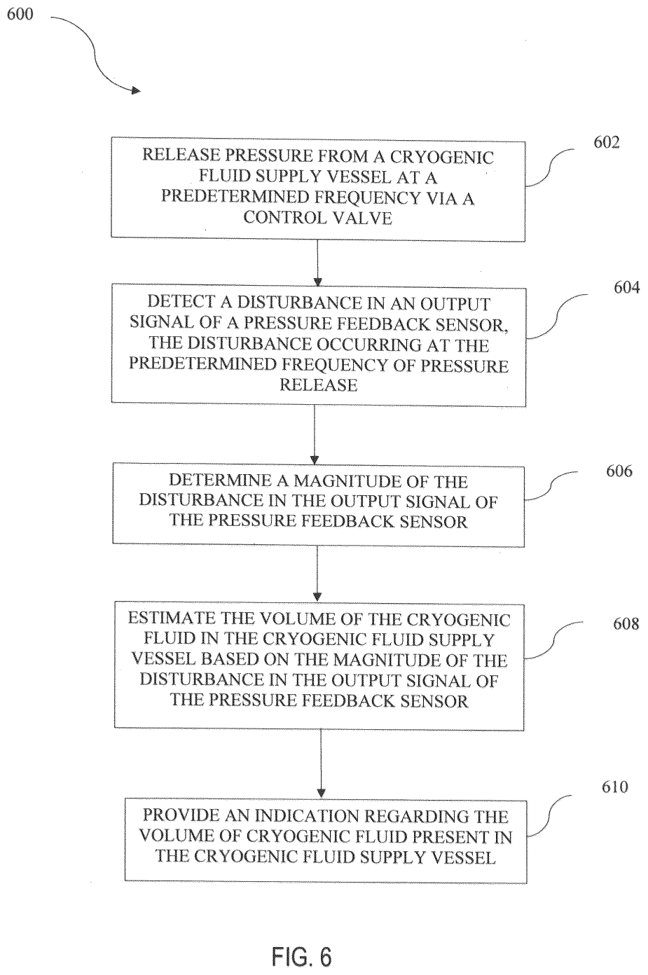

Referring now to FIG. 6, an example workflow 600 for a cryogen delivery procedure, utilized, for example, by the cryogen delivery system 100 of FIG. 1, is shown. At 602, pressure is released for a cryogenic fluid supply vessel 104A, which is coupled to the cryogen delivery system 100, at a predetermined frequency via a control value 206. In some embodiments, for example, as discussed above with reference to FIGS. 2 and 3, to regulate a pressure of the cryogenic fluid supply vessel 104A throughout a procedure (e.g., a cryoablation procedure), a cryoablation control board (CCB) 202 of a control system 200 is configured to release a pressure P_{D} from the cryogenic fluid supply vessel 104A by opening/closing (or toggling) the control valve 206 at a frequency of about 3.33 Hz.

At 604, a disturbance D in an output signal of a pressure feedback sensor 208, which occurs at the predetermined frequency of pressure release, is detected, for example by the CCB 202. As discussed above, with further reference to FIGS. 5A, 5B, and 6, whenever the CCB 202 toggles the control valve 206 (e.g., by pulsing a voltage command V_{CV}, for example, by about 3.33 Hz), there is a ripple in the measured pressure output from the cryogenic fluid supply vessel 104A (i.e., the pressure P_{D}). In accordance with the present disclosure, the CCB 202 is configured to detect this disturbance (or ripple) D in the output signal of the pressure feedback sensor 208 (which corresponds to the rate of pressure release from the cryogenic fluid supply vessel 104A).

At 606, a magnitude M of the disturbance D in the output signal of the pressure feedback sensor 208 is determined. As discussed above, in some embodiments, the CCB 202 is further configured to determine the magnitude M of a respective disturbance D by extracting the respective disturbance D from the output signal of the pressure feedback sensor 208 and applying one or more of a min/max detection algorithm and/or a discrete Fourier transform (DFT) to the extracted disturbance D.

At 608, the volume of the cryogenic fluid in the cryogenic fluid supply vessel 104A is estimated based on the magnitude M of the disturbance D in the output signal of the pressure feedback sensor 208. As further discussed above, the present disclosure recognizes that a magnitude M of a respective disturbance D correlates directly with a volume of cryogenic fluid in the fluid supply vessel 104A, such that as the volume of cryogenic fluid in the fluid supply vessel 104A decreases a magnitude M of a disturbance D in the output signal of the pressure feedback sensor 208 decreases. In this manner, the CCB 202 can be configured to estimate the volume of a cryogenic fluid, such as, for example liquid nitrogen (LN₂), in the fluid supply vessel 104A at any point during a procedure by correlating one or more of the determined magnitudes M (i.e., of the detected disturbances D) with preestablished volume levels, via, for example, a look-up table or other known correlation method.

At 610, an indication regarding the estimated volume of cryogenic fluid (e.g., LN₂) in the cryogenic fluid supply vessel 104A is provided, for example, to an operator of the cryogen delivery system 100. For example, as discussed above with reference to FIG. 4, the CCB 202 may provide an indication to the operator by providing information, via, for example, the display 108, 424, regarding an available volume, a maximum procedure run time, and/or a binary go/no-go indicator that indicates whether the amount of cryogen present/available is sufficient to complete a procedure chosen by the operator. The display 108, 424 (e.g., via the cryogen status indicator area 436) can be configured to display the information regarding the available volume of cryogen via text, colors, icons, warning lights (flashing or steady state) on the display or on a portion of the console (e.g., console 100 shown in FIG. 1), or any combination thereof. The visual indicators may be combined with audio indicators such as speech reciting displayed text indicators, beeping, or other audible indicators.

The workflow 600 can, for example, be utilized while ablating a target tissue during a cryoablation procedure. For example, the cryoablation procure may include inserting a cryogenic probe 106 of the cryogen delivery system 100 into a treatment region of a patient. In some embodiments, for example, this may comprise inserting the cryogenic probe 106 into breast tissue of a patient. In other embodiments, the probe may be used as a cryosurgical tool in the fields of general surgery, dermatology, thoracic surgery, gynecology, oncology, proctology, and urology. As discussed above, the cryogenic probe 106 is in fluid communication with a cryogenic fluid supply vessel 104A containing liquid nitrogen. A constant pressure is then supplied to the cryogenic fluid supply vessel 104A via a compressor of the cryogen delivery system 100 during a freeze cycle of the cryoablation procedure to supply the liquid nitrogen to the cryogenic probe 106. For example, the liquid nitrogen may be supplied to the probe 106 during a first freeze cycle, a second freeze, and/or a later freeze cycle of the cryogen delivery system 100.

As above, during a respective freeze cycle, a pressure in the cryogenic fluid supply vessel 104A is regulated by releasing pressure from the cryogenic fluid supply vessel, and an amount of liquid nitrogen remaining in the cryogenic fluid supply vessel during a respective freeze cycle can be monitored by this pressure release. For example, as discussed above, the CCB 202 can detect a ripple in an output signal of a pressure feedback sensor 208 of the cryogen delivery system 100, the ripple occurring at a frequency corresponding to a rate of pressure release from the cryogenic fluid supply vessel. The CCB 202 can then determine a magnitude M of the ripple in the output signal of the pressure feedback sensor 208 and based on the magnitude M of the ripple in the output signal of the pressure feedback sensor 208, estimate an amount of the liquid nitrogen remaining in the cryogenic fluid supply vessel 104A. In this manner, based on an estimated amount of liquid nitrogen remaining in the cryogenic fluid supply vessel 104A at an end of a respective freeze cycle, the CCB 202 can determine whether there is enough liquid nitrogen to initiate another freeze cycle. Thus, the CCB, for example, may be configured to only initiate a second freeze cycle after confirming that the estimated amount of liquid nitrogen remaining in the cryogenic fluid supply vessel 104A at an end of the first freeze cycle is sufficient to complete the second freeze cycle.

The methods and systems according to the present disclosure can be used, for example, for cryoablation procedures used for treatment of both benign and cancerous tumors (e.g., to freeze cancerous legions and benign tumors). In one example according to the disclosure of U.S. Pat. No. 8,092,448, incorporated above, a fibroadenoma with a major axis of 3.1 to 3.5 cm diameter is treated by two cycles of freezing, each consisting of 6 minutes of freezing, with 10 minutes of passive thawing between the freezing cycles. As an additional example, a fibroadenoma of 3.6 to 4.0 cm diameter is treated by two cycles of freezing, each consisting of 8 minutes of freezing, with 10 minutes of passive thawing between the freezing cycles. Further examples for fibroadenomas of different sizes and shapes are provided in U.S. Pat. No. 8,092,448.

The methods and systems according to the present disclosure can increase the efficiency and efficacy of cryoablation procedures by providing useful information to the operator of the cryogen delivery system regarding the amount of cryogen present in a cryogenic fluid supply vessel at any point during a cryogenic procedure and are not limited to providing information only at the start of a procedure or during a pause in a procedure.

### Examples

Illustrative examples of the systems methods described herein are provided below. An embodiment of the system and method described herein may include any one or more, and any combination of, the clauses described below:

Clause 1. A method for estimating an amount of cryogenic fluid in a cryogenic fluid supply vessel, the method comprising:
releasing pressure from the cryogenic fluid supply vessel at a predetermined frequency via a control valve;
detecting a disturbance in an output signal of a pressure feedback sensor, the disturbance occurring at the predetermined frequency;
determining a magnitude of the disturbance in the output signal of the pressure feedback sensor; and
estimating the amount of the cryogenic fluid in the cryogenic fluid supply vessel based on the magnitude of the disturbance in the output signal of the pressure feedback sensor.

Clause 2. The method of clause 1, wherein:
releasing the pressure from the cryogenic fluid supply vessel at the predetermined frequency comprises releasing the pressure from the cryogenic fluid supply vessel at a rate of about 3.33 Hz, and
detecting the disturbance in the output signal of the pressure feedback sensor comprises detecting a 3.33 Hz disturbance in the output signal of the pressure feedback sensor.

Clause 3. The method of clause 1 or clause 2, wherein releasing the pressure from the cryogenic fluid supply vessel at the predetermined frequency via the control valve comprises operating a pressure regulator to open and close the control valve.

Clause 4. The method of any one of clauses 1-3, further comprising, prior to releasing the pressure from the cryogenic fluid supply vessel, supplying a constant pressure to the cryogenic fluid supply vessel via a compressor.

Clause 5. The method of clause 4, wherein releasing the pressure from the cryogenic fluid supply vessel comprises releasing a combined pressure from the cryogenic fluid supply vessel, the combined pressure including the supplied constant pressure and a fluid pressure from the cryogenic fluid in the cryogenic fluid supply vessel.

Clause 6. The method of clause 5, wherein releasing the combined pressure from the cryogenic fluid supply vessel comprises regulating the pressure in the cryogenic fluid supply vessel to a pre-established level.

Clause 7. The method of clause 6, wherein regulating the pressure in the cryogenic fluid supply vessel to the pre-established level comprises regulating the pressure in the cryogenic fluid supply vessel to about 30 psi.

Clause 8. The method of any one of clauses 1-7, wherein estimating the amount of the cryogenic fluid in the cryogenic fluid supply vessel comprises estimating a volume of the cryogenic fluid in the cryogenic fluid supply vessel during a cryogenic procedure utilizing the cryogenic fluid supply vessel.

Clause 9. The method of any one of clauses 1-8, wherein determining the magnitude of the disturbance in the output signal of the pressure feedback sensor comprises extracting the disturbance from the output signal and applying a min/max detection algorithm to the disturbance.

Clause 10. The method of any one of clauses 1-8, wherein determining the magnitude of the disturbance in the output signal of the pressure feedback sensor comprises extracting the disturbance from the output signal and applying a discrete Fourier transform (DFT) to the disturbance.

Clause 11. The method of any one of clauses 1-10, wherein the cryogenic fluid in the cryogenic fluid supply vessel comprises liquid nitrogen, and wherein estimating the amount of the cryogenic fluid in the cryogenic fluid supply vessel based on the magnitude of the disturbance in the output signal of the pressure feedback sensor comprises estimating the amount of the liquid nitrogen in the cryogenic fluid supply vessel based on the magnitude of the disturbance in the output signal of the pressure feedback sensor.

Clause 12. The method of any one of clauses 1-11, further comprising providing an indication regarding the amount of cryogenic fluid present in the cryogenic fluid supply vessel.

Clause 13. The method of clause 12, wherein providing an indication regarding the amount of cryogenic fluid present in the cryogenic fluid supply vessel comprises one or more of a visible indication and an audible indication.

Clause 14. The method of clause 13, wherein providing an indication regarding the amount of cryogenic fluid present in the cryogenic fluid supply vessel includes displaying the indication on a display associated with a cryogen delivery system.

Clause 15. The method of clause 14, wherein providing the indication regarding the amount of cryogenic fluid present in the cryogenic fluid supply vessel comprises providing an indication that the amount of cryogenic fluid present is sufficient to complete a freezing cycle of the cryogen delivery system and/or providing an indication that the amount of cryogenic fluid present is insufficient to complete a freezing cycle of the cryogen delivery system.

Clause 16. The method of clause 14, wherein providing the indication regarding the amount of cryogenic fluid present in the cryogenic fluid supply vessel comprises providing an indication that the amount of cryogenic fluid present is sufficient to complete a treatment cycle of the cryogen delivery system and/or providing an indication that the amount of cryogenic fluid present is insufficient to complete a treatment cycle of the cryogen delivery system.

Clause 17. The method of any one of clauses 1-16, wherein:
detecting a disturbance in the output signal of the pressure feedback sensor comprises detecting a ripple in the output signal of the pressure feedback sensor, and
determining the magnitude of the disturbance in the output signal of the pressure feedback sensor comprising calculating a magnitude of the ripple in the output signal of the pressure feedback sensor.

Clause 18. The method of clause 17, wherein the magnitude of the ripple is proportional to a volume of the cryogenic fluid in the cryogenic fluid supply vessel.

Clause 19. The method of clause 1, wherein estimating the amount of the cryogenic fluid in the cryogenic fluid supply vessel comprises automatically estimating the amount of the cryogenic fluid in the cryogenic fluid supply vessel prior to or during a cryogenic procedure.

Clause 20. The method of clause 1, wherein estimating the amount of the cryogenic fluid in the cryogenic fluid supply vessel comprises initiating a request to estimate the amount of the cryogenic fluid in the cryogenic fluid supply vessel prior to or during a cryogenic procedure.

Clause 21. A method for estimating an amount of liquid nitrogen in a cryogenic fluid supply vessel during a cryoablation procedure, the method comprising:
supplying a constant pressure to the cryogenic fluid supply vessel via a compressor of a cryogen delivery system;
regulating a pressure in the cryogenic fluid supply vessel by releasing the pressure from the cryogenic fluid supply vessel, the pressure from the cryogenic fluid supply vessel comprising a combination of the constant pressure from the compressor and a fluid pressure from a volume of liquid nitrogen in the cryogenic fluid supply vessel;
detecting a ripple in an output signal of a pressure feedback sensor of the cryogen delivery system, the ripple occurring at a frequency corresponding to a rate of pressure release from the cryogenic fluid supply vessel;
determining a magnitude of the ripple in the output signal of the pressure feedback sensor; and
estimating a remaining amount of the liquid nitrogen in the cryogenic fluid supply vessel based on the magnitude of the ripple in the output signal of the pressure feedback sensor.

Clause 22. The method of clause 21, wherein regulating the pressure in the cryogenic fluid supply vessel comprises releasing the pressure from the cryogenic fluid supply vessel at a predetermined frequency by operating a pressure regulator to open and close a control valve at the predetermined frequency.

Clause 23. The method of clause 22, wherein:
releasing the pressure from the cryogenic fluid supply vessel at the predetermined frequency comprises releasing the pressure from the cryogenic fluid supply vessel at a rate of about 3.33 Hz, and
detecting the ripple in the output signal of the pressure feedback sensor comprises detecting a 3.33 Hz ripple in the output signal of the pressure feedback sensor.

Clause 24. The method of clause 23, wherein regulating the pressure in the cryogenic fluid supply vessel comprises regulating the pressure in the cryogenic fluid supply vessel to about 30 psi.

Clause 25. The method of any one of clauses 21-24, wherein determining the magnitude of the ripple in the output signal of the pressure feedback sensor comprises applying a min/max detection algorithm to the ripple.

Clause 26. The method of any one of clauses 21-24, wherein determining the magnitude of the ripple in the output signal of the pressure feedback sensor comprises applying a discrete Fourier transform (DFT) to the ripple.

Clause 27. The method of any one of clauses 21-26, further comprising providing an indication regarding the remaining amount of liquid nitrogen present in the cryogenic fluid supply vessel.

Clause 28. The method of clause 27, wherein providing an indication regarding the remaining amount of liquid nitrogen present in the cryogenic fluid supply vessel comprises one or more of a visible indication and an audible indication.

Clause 29. The method of clause 28, wherein providing an indication regarding the remaining amount of liquid nitrogen in the cryogenic fluid supply vessel includes displaying the indication on a display associated with the cryogen delivery system.

Clause 30. The method of any one of clauses 27-29, wherein providing the indication regarding the remaining amount of liquid nitrogen present in the cryogenic fluid supply vessel comprises providing an indication that the remaining amount of liquid nitrogen present is sufficient to complete a freezing cycle of the cryogen delivery system and/or providing an indication that the remaining amount of cryogenic fluid present is insufficient to complete a freezing cycle of the cryogen delivery system.

Clause 31. The method of any one of clauses 27-29, wherein providing the indication regarding the remaining amount of liquid nitrogen present in the cryogenic fluid supply vessel comprises providing an indication that the remaining amount of liquid nitrogen present is sufficient to complete a treatment cycle of the cryogen delivery system and/or providing an indication that the remaining amount of cryogenic fluid present is insufficient to complete a treatment cycle of the cryogen delivery system.

Clause 32. A method of ablating a target tissue during a cryoablation procedure, the method comprising:
inserting a cryogenic probe of a cryogen delivery system into a treatment region of a patient, the cryogenic probe being in fluid communication with a cryogenic fluid supply vessel containing liquid nitrogen;
supplying a constant pressure to the cryogenic fluid supply vessel via a compressor of the cryogen delivery system during a freeze cycle of the cryoablation procedure to supply the liquid nitrogen to the cryogenic probe;
regulating a pressure in the cryogenic fluid supply vessel by releasing the pressure from the cryogenic fluid supply vessel, the pressure from the cryogenic fluid supply vessel comprising a combination of the constant pressure from the compressor and a fluid pressure from a volume of liquid nitrogen in the cryogenic fluid supply vessel; and
monitoring an amount of liquid nitrogen remaining in the cryogenic fluid supply vessel during the freeze cycle by:
detecting a ripple in an output signal of a pressure feedback sensor of the cryogen delivery system, the ripple occurring at a frequency corresponding to a rate of pressure release from the cryogenic fluid supply vessel;
determining a magnitude of the ripple in the output signal of the pressure feedback sensor; and
based on the magnitude of the ripple in the output signal of the pressure feedback sensor, estimating an amount of the liquid nitrogen remaining in the cryogenic fluid supply vessel.

Clause 33. The method of clause 32, wherein:
supplying the constant pressure to the cryogenic fluid supply vessel via the compressor of the cryogen delivery system during the freeze cycle of the cryoablation procedure comprises supplying a constant pressure to the cryogenic fluid supply vessel via the compressor of the cryogen delivery system during a first freeze cycle of the cryoablation procedure, and
monitoring the amount of liquid nitrogen remaining in the cryogenic fluid supply vessel during the freeze cycle comprises monitoring an amount of liquid nitrogen remaining in the cryogenic fluid supply vessel during the first freeze cycle.

Clause 34. The method of clause 33, wherein:
supplying the constant pressure to the cryogenic fluid supply vessel via the compressor of the cryogen delivery system during the freeze cycle of the cryoablation procedure comprises supplying a constant pressure to the cryogenic fluid supply vessel via the compressor of the cryogen delivery system during a second freeze cycle of the cryoablation procedure, the second freeze cycle occurring at a later time than the first freeze cycle, and
monitoring the amount of liquid nitrogen remaining in the cryogenic fluid supply vessel during the freeze cycle comprises monitoring an amount of liquid nitrogen remaining in the cryogenic fluid supply vessel during the second freeze cycle.

Clause 35. The method of clause 32, further comprising, based on an estimated amount of liquid nitrogen remaining in the cryogenic fluid supply vessel at an end of the first freeze cycle, initiating a second freeze cycle of the cryoablation procedure.

Clause 36. The method of clause 32, wherein inserting the cryogenic probe of the cryogen delivery system into the treatment region of the patient comprises inserting the cryogenic probe into breast tissue of the patient.

Clause 37. The method of clause 32, wherein inserting the cryogenic probe of the cryogen delivery system into the treatment region of the patient comprises inserting the cryogenic probe in the treatment region to freeze a cancerous lesion.

Clause 38. The method of clause 32, wherein inserting the cryogenic probe of the cryogen delivery system into the treatment region of the patient comprises inserting the cryogenic probe in the treatment region to freeze a benign tumor.

The method for estimating a volume of cryogenic fluid in a cryogenic fluid supply vessel of any one of clauses 1-21 can be practiced in conjunction with the methods of any one of clauses 22-38.

The method for estimating a volume of cryogenic fluid in a cryogenic fluid supply vessel of any one of clauses 22-31 can be practiced in conjunction with the methods of any one of clauses 1-21 and 32-38.

The method of ablating a target tissue during a cryoablation procedure of any one of clauses 32-38 can be practiced in conjunction with the methods of any one of clauses 1-21 and 22-31.

This disclosure described some examples of the present technology with reference to the accompanying drawings, in which some of the possible examples were shown. Other aspects can, however, be embodied in many different forms and should not be construed as limited to the examples set forth herein. Rather, these examples were provided so that this disclosure was thorough and complete and fully conveyed the scope of the possible examples to those skill in the art.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

This description and the accompanying drawings that illustrate exemplary embodiments should not be taken as limiting. Various mechanical, compositional, structural, electrical, and operational changes may be made without departing from the scope of this description and the claims, including equivalents. In some instances, well-known structures and techniques have not been shown or described in detail so as not to obscure the disclosure. Furthermore, elements and their associated features that are described in detail with reference to one embodiment may, whenever practical, be included in other embodiments in which they are not specifically shown or described. For example, if an element is described in detail with reference to one embodiment and is not described with reference to a second embodiment, the element may nevertheless be included in the second embodiment.

It is noted that, as used herein, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Further, this description's terminology is not intended to limit the disclosure. For example, spatially relative terms—such as “beneath,” “below,” “lower,” “above,” “upper,” “forward,” “front,” “behind,” and the like—may be used to describe one element's or feature's relationship to another element or feature as illustrated in the orientation of the figures. These spatially relative terms are intended to encompass different positions and orientations of a device in use or operation in addition to the position and orientation shown in the figures. For example, if a device in the figures is inverted, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly.

Further modifications and alternative embodiments will be apparent to those of ordinary skill in the art in view of the disclosure herein. For example, the methods and systems may include additional components that were omitted from the diagrams and description for clarity of operation. Accordingly, this description is to be construed as illustrative only and is for the purpose of teaching those skilled in the art the general manner of carrying out the methods and systems of the present disclosure. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the scope of the present disclosure.

It is to be understood that the particular examples and embodiments set forth herein are non-limiting, and modifications to structure, dimensions, materials, and methodologies may be made without departing from the scope of the present disclosure. Other embodiments in accordance with the present disclosure will be apparent to those skilled in the art from consideration of the specification and practice of the invention disclosed herein. It is intended that the specification and examples be considered as exemplary only, with being entitled to their full breadth of scope, including equivalents.

What is claimed is:

1. A method for estimating an amount of cryogenic fluid in a cryogenic fluid supply vessel, the method comprising:
releasing pressure from the cryogenic fluid supply vessel at a predetermined frequency via a control valve;
detecting a disturbance in an output signal of a pressure feedback sensor, the disturbance occurring at the predetermined frequency;
determining a magnitude of the disturbance in the output signal of the pressure feedback sensor; and
estimating the amount of the cryogenic fluid in the cryogenic fluid supply vessel based on the magnitude of the disturbance in the output signal of the pressure feedback sensor.

2. The method of claim 1, wherein:
releasing the pressure from the cryogenic fluid supply vessel at the predetermined frequency comprises releasing the pressure from the cryogenic fluid supply vessel at a rate of about 3.33 Hz, and
detecting the disturbance in the output signal of the pressure feedback sensor comprises detecting a 3.33 Hz disturbance in the output signal of the pressure feedback sensor.

3. The method of claim 1, wherein releasing the pressure from the cryogenic fluid supply vessel at the predetermined frequency via the control valve comprises operating a pressure regulator to open and close the control valve.

4. The method of claim 1, further comprising, prior to releasing the pressure from the cryogenic fluid supply vessel, supplying a constant pressure to the cryogenic fluid supply vessel via a compressor.

5. The method of claim 4, wherein releasing the pressure from the cryogenic fluid supply vessel comprises releasing a combined pressure from the cryogenic fluid supply vessel, the combined pressure including the supplied constant pressure and a fluid pressure from the cryogenic fluid in the cryogenic fluid supply vessel.

6. The method of claim 5, wherein releasing the combined pressure from the cryogenic fluid supply vessel comprises regulating the pressure in the cryogenic fluid supply vessel to a pre-established level.

7. The method of claim 1, wherein estimating the amount of the cryogenic fluid in the cryogenic fluid supply vessel comprises estimating a volume of the cryogenic fluid in the cryogenic fluid supply vessel during a cryogenic procedure utilizing the cryogenic fluid supply vessel.

8. The method of claim 1, wherein determining the magnitude of the disturbance in the output signal of the pressure feedback sensor comprises extracting the disturbance from the output signal and applying a min/max detection algorithm and/or a discrete Fourier transform (DFT) to the disturbance.

9. The method of claim 1, wherein the cryogenic fluid in the cryogenic fluid supply vessel comprises liquid nitrogen, and wherein estimating the amount of the cryogenic fluid in the cryogenic fluid supply vessel based on the magnitude of the disturbance in the output signal of the pressure feedback sensor comprises estimating the amount of the liquid nitrogen in the cryogenic fluid supply vessel based on the magnitude of the disturbance in the output signal of the pressure feedback sensor.

10. The method of claim 1, further comprising providing an indication regarding the amount of cryogenic fluid present in the cryogenic fluid supply vessel.

11. The method of claim 10, wherein providing the indication regarding the amount of cryogenic fluid present in the cryogenic fluid supply vessel comprises providing an indication that the amount of cryogenic fluid present is sufficient to complete a freezing cycle and/or a treatment cycle of a cryogen delivery system utilizing the cryogenic fluid supply vessel and/or providing an indication that the amount of cryogenic fluid present is insufficient to complete a freezing cycle and/or a treatment cycle of the cryogen delivery system.

12. The method of claim 1, wherein:
detecting a disturbance in the output signal of the pressure feedback sensor comprises detecting a ripple in the output signal of the pressure feedback sensor, and
determining the magnitude of the disturbance in the output signal of the pressure feedback sensor comprising calculating a magnitude of the ripple in the output signal of the pressure feedback sensor.

13. The method of claim 12, wherein the magnitude of the ripple is proportional to a volume of the cryogenic fluid in the cryogenic fluid supply vessel.

14. A method for estimating an amount of liquid nitrogen in a cryogenic fluid supply vessel during a cryoablation procedure, the method comprising:
supplying a constant pressure to the cryogenic fluid supply vessel via a compressor of a cryogen delivery system;
regulating a pressure in the cryogenic fluid supply vessel by releasing the pressure from the cryogenic fluid supply vessel, the pressure from the cryogenic fluid supply vessel comprising a combination of the constant pressure from the compressor and a fluid pressure from a volume of liquid nitrogen in the cryogenic fluid supply vessel;
detecting a ripple in an output signal of a pressure feedback sensor of the cryogen delivery system, the ripple occurring at a frequency corresponding to a rate of pressure release from the cryogenic fluid supply vessel;
determining a magnitude of the ripple in the output signal of the pressure feedback sensor; and
estimating a remaining amount of the liquid nitrogen in the cryogenic fluid supply vessel based on the magnitude of the ripple in the output signal of the pressure feedback sensor.

15. The method of claim 14, wherein regulating the pressure in the cryogenic fluid supply vessel comprises releasing the pressure from the cryogenic fluid supply vessel at a predetermined frequency by operating a pressure regulator to open and close a control valve at the predetermined frequency to regulate the pressure in the cryogenic fluid supply vessel to about 30 psi.

16. A method of ablating a target tissue during a cryoablation procedure, the method comprising:
inserting a cryogenic probe of a cryogen delivery system into a treatment region of a patient, the cryogenic probe being in fluid communication with a cryogenic fluid supply vessel containing liquid nitrogen;
supplying a constant pressure to the cryogenic fluid supply vessel via a compressor of the cryogen delivery system during a freeze cycle of the cryoablation procedure to supply the liquid nitrogen to the cryogenic probe;
regulating a pressure in the cryogenic fluid supply vessel by releasing the pressure from the cryogenic fluid supply vessel, the pressure from the cryogenic fluid supply vessel comprising a combination of the constant pressure from the compressor and a fluid pressure from a volume of liquid nitrogen in the cryogenic fluid supply vessel; and
monitoring an amount of liquid nitrogen remaining in the cryogenic fluid supply vessel during the freeze cycle by:
detecting a ripple in an output signal of a pressure feedback sensor of the cryogen delivery system, the ripple occurring at a frequency corresponding to a rate of pressure release from the cryogenic fluid supply vessel;
determining a magnitude of the ripple in the output signal of the pressure feedback sensor; and
based on the magnitude of the ripple in the output signal of the pressure feedback sensor, estimating an amount of the liquid nitrogen remaining in the cryogenic fluid supply vessel.

17. The method of claim 16, wherein:
supplying the constant pressure to the cryogenic fluid supply vessel via the compressor of the cryogen delivery system during the freeze cycle of the cryoablation procedure comprises supplying a constant pressure to the cryogenic fluid supply vessel via the compressor of the cryogen delivery system during a first freeze cycle of the cryoablation procedure, and
monitoring the amount of liquid nitrogen remaining in the cryogenic fluid supply vessel during the freeze cycle comprises monitoring an amount of liquid nitrogen remaining in the cryogenic fluid supply vessel during the first freeze cycle.

18. The method of claim 17, wherein:
supplying the constant pressure to the cryogenic fluid supply vessel via the compressor of the cryogen delivery system during the freeze cycle of the cryoablation procedure comprises supplying a constant pressure to the cryogenic fluid supply vessel via the compressor of the cryogen delivery system during a second freeze cycle of the cryoablation procedure, the second freeze cycle occurring at a later time than the first freeze cycle, and
monitoring the amount of liquid nitrogen remaining in the cryogenic fluid supply vessel during the freeze cycle comprises monitoring an amount of liquid nitrogen remaining in the cryogenic fluid supply vessel during the second freeze cycle.

19. The method of claim 17, further comprising, based on an estimated amount of liquid nitrogen remaining in the cryogenic fluid supply vessel at an end of the first freeze cycle, initiating a second freeze cycle of the cryoablation procedure.

20. The method of claim 16, wherein inserting the cryogenic probe of the cryogen delivery system into the treatment region of the patient comprises inserting the cryogenic probe into breast tissue of the patient or inserting the cryogenic probe in the treatment region to freeze a cancerous lesion and/or a benign tumor.
